(19) United States
(12) Patent Application Publication
(10) Pub. No.: US 2015/0148724 A1
(43) Pub. Date: May 28, 2015

(54) ENHANCED THERMOPLASTIC MATERIALS FOR ORTHOSES AND PROSTHESES

(71) Applicant: Gary George Bedard, San Mateo, CA (US)

(72) Inventor: Gary George Bedard, San Mateo, CA (US)

(21) Appl. No.: 14/551,488

(22) Filed: Nov. 24, 2014

Related U.S. Application Data
(60) Provisional application No. 61/908,298, filed on Nov. 25, 2013.

(51) Int. Cl. A61F 5/01; B29C 51/10; A61L 27/34; B29C 51/00; A61F 2/60; A61L 27/44

(52) U.S. Cl. CPC A61F 5/0127; A61F 2/60; A61L 27/44; A61L 27/34; B29C 51/002; B29C 51/10; A61F 2002/608; B29K 2307/04
USPC 602/7; 623/27; 428/339; 428/473.5; 428/523; 428/517; 428/518; 428/412; 428/483; 428/516; 264/571

(57) ABSTRACT

An improved thermoplastic material for orthoses and prostheses. This material comprises at least one resin layer of polymeric resin, optionally reinforced with multiple layers or internal fibers, designed to be compatible with vacuum thermoforming, with thickness between 3/64 inches and ¾ inches. The material additionally comprises at least one and optionally two extremely thin cap layers with thickness between 2/1000 and 12/1000 inches, at least one of which is intended for improved compatibility with human skin, and the other of which can be configured for various cosmetic or UV protection purposes. Methods of producing this material, and various orthotic and prosthetic devices using this material, are also taught.

### CROSS REFERENCE TO RELATED APPLICATIONS

This application claims the priority benefit of U.S. provisional patent application 61/908,298 “ENHANCED THERMOPLASTIC MATERIALS FOR ORTHOSES AND PROSTHESES”, inventor Gary George Bedard, filed Nov. 25, 2013; the entire contents of this application is incorporated herein by reference.

### BACKGROUND OF THE INVENTION

1. Field of the Invention

This invention relates to orthoses and prostheses, and more particularly to materials and methods to fabricate and reinforce the clinical devices with a multi-element thermoplastic sheetstock.

2. Description of the Related Art

Since the early 1970s, heavy gauge thermoplastic sheetstock has been used to produce custom orthoses (orthopedic braces or appliances) and prostheses (artificial limbs) and components thereof, usually by way of a vacuum forming process. The methodology of clinical vacuum forming with thermoplastic sheetstock was first explored at numerous medical centers around the United States (Vacuum Forming of Plastics in Prosthetics and Orthotics, A. Bennett Wilson Jr., Orthotics and Prosthetics, Vol. 28, No 1, pp. 12-20, March 1974). The advent of “total contact” orthoses offered an alternative to historical leather and metal fabrication of conventional orthoses (Thermoformed Ankle-Foot Orthoses Stills. M, Orthotics & Prosthetics, Vol. 29, No 4, pp. 41-51, December 1975).

Clinical products made in this fashion offer good durability, hypoallergenic patient contact surfaces and the ability to mold a product that is very intimate with the body segment of the patient that requires external support alignment or stabilization.

A custom orthoses (orthosis) or (prosthesis) prosthetic device (alternatively orthotic or prosthesis device, orthotic device, prosthetic device, clinical product, custom clinical product or in the plural orthoses and prostheses) is often fabricated by first making mold of the portion of the body over which the device is intended to be worn. Current practice is to cast the body part with plaster of Paris bandages or synthetic casting tapes. Once cured and removed from the body, the plaster or synthetic cast is called a negative model. To turn this into a positive model, the negative model is then dammed off and filled with slurry of plaster of Paris, which is then allowed to harden. The negative cast is then removed from the surface of the positive model, and a plaster rendition of the body segment is provided as an unimproved positive model. The positive model is then improved for various clinical implications offering correction, alignment, stabilization and protection. The improved positive model is then used as a mold to fabricate a custom clinical product, specifically designed to fit that particular patient's body part.

The human form is a variable commodity, and changes in volume and shape with respect to growth, weight gain and underlying clinical patho-etiologies and pathomechanics which frequently occur. As a result, orthotic or prosthetic devices frequently need to be adjusted in the post delivery and patient use stage. This is usually done by a practitioner orthotist or prosthetist (i.e. an orthosis or prosthesis device expert) as the patient's body undergoes change.

In order to meet this need for easy adjustment, the custom clinical product is often fabricated using thermoplastic sheetstock. This material has certain advantages for forming orthotic or prosthetic devices because its shape can be easily adjusted by heat molding (viscoelastic remodeling) the material in the post delivery and patient use stage.

To make these adjustments, a practitioner will often make spot or isolated changes in the device through the use of a heat gun. A heat gun produces a directed stream of heated air. The delivery of the heated air can be directed to a spot in the clinical device that may be causing discomfort to the patient due to pressure and laminar sheer against the skin and underlying skeletal prominence. The heated spot can be re-contoured through viscoelastic remodeling of the thermoplastic sheetstock, with no reduction in the strength of the cooled thermoplastic material.

Orthotic fabrication is typically done under very low pressures and at temperatures that are roughly around the melt temperature of the thermoplastic material. Here, for example, thermoplastic sheetstock that has been heated to roughly melt temperature may simply be grasped by hand (using insulated gloves) at the four corners of the sheet, draped over the plaster of Paris positive mold of the body part, and deformed or reformed to fit the mold by low pressure vacuum forming. To do this low pressure (open atmosphere) vacuum forming, a vacuum is applied to the internal space between the thermoplastic sheetstock and the positive model which removes any residual air in the captured space. The force of the ambient (atmospheric) air pressure then intimately molds the thermoplastic sheetstock to the surface of the positive model. This forming process is facilitated by the fact that thermoplastic sheetstock is generally self-adhesive at melt temperature, and will seal to itself during this process, eliminating the need for any accessory adhesive for the airtight seal around the positive model. See for example, U.S. Pat. No. 2,978,376 (Hulse).

Typical thermoplastic resins used for thermoforming may include ABS, Kydex®, Lexan®, VIVAK®, TPE, PVC, polystyrene and numerous other generic and proprietary resins. Thermopolymer polyolefin resins in the form of polyethylene (PE) were first developed in 1934 by ICI in the United Kingdom. Dupont opened the first PE plant to support the war effort in 1943. Polypropylene, another polyolefin variant, became suitable for heavy gauge thermoforming in the mid-1950s. Isotatic polypropylene is the most common type of polypropylene sheetstock that is used for vacuum thermoforming.

Prior art on the construction of orthosis or prosthesis devices includes U.S. Pat. No. 1,232,899 (De Puy), U.S. Pat. No. 3,916,886 (Rogers), and U.S. Pat. No. 4,289,122 (Mason & Vuletich).

Although pure homopolymer sheetstock thus has many advantages for these applications, this material also has a number of significant disadvantages.

One disadvantage of using a pure homopolymer sheetstock for cut sheet heavy gauge vacuum thermoforming is the relative lack of sheet strength at melt temperature. Sheetstock that is heated just a small degree over the recommended temperature molding range for the particular sag strength of the specific resin formulation can undergo a sharp change from high viscosity to low viscosity, and as a result droop very quickly in the transfer from the non-stick oven tray to the positive model, forming regions of non-uniform thickness.

This sharp change in viscosity is a particular problem for sheetstock made from homopolymer polypropylene and copolymer polypropylene. Overly hot thermoplastic sheetstock can rapidly stretch during the hand-held manipulation process. This can form thin regions in the material, resulting in a final product that might not be stiff enough to be suitable as a clinical device, resulting in wasted materials and effort.

Another drawback of using pure homogenous thermoplastic sheetstock is that the final orthoses and prostheses made from such un-reinforced homogeneous thermoplastic sheetstock have a tendency to further deform or reform with use. That is, although the orthoses or prosthesis may originally fit the patient well, with use the devices will further deform or reform, and gradually fit the patient less well. This gradual deformation is an example of “creep” or, for a clinical orthosis or prosthesis device, this creep can be termed “clinical creep”. Thus an orthosis or prosthesis that might originally fit the patient well will, with use, end up fitting poorly.

Creep is a common characteristic of thermoplastic materials, especially semi-crystalline materials such as polypropylene and polyethylene and their variants. The polymer chains comprising the molecular structure of these resins are not chemically cross linked. As a result, the polymer chains will continue to move and allow the product to change shape over time, even when the use of the product is within the normal temperature service range for the particular resin. Creep is thus due to the natural viscoelastic properties of thermoplastic materials. Creep typically occurs in the amorphous area of the polymer chain structure and not within the crystalline area of a polyolefin resin.

Clinical creep or “creep” is thus a manifestation of the viscoelastic change that occurs in a clinical device fabricated from a thermoplastic material that will change shape due to the influence of gait forces that are applied to the device as well as the increase in temperature of the device from absorption of heat from contact with the human body. Clinical creep is a disadvantage in a lower extremity orthosis especially, because the foot and ankle complex requires the maintenance of optimal skeletal alignment, support and stabilization.

Even the patient's body heat can change the viscoelastic properties of thermoplastic sheetstock, and normal body temperature may raise the temperature of the device, which will accelerate clinical creep. The change in shape in the clinical product from clinical creep may decrease the efficiency of the device in the long term and lessen the useful life of the product. The polyolefin family of resins, which includes homopolymer polypropylene, co-polymer polypropylene and the polyethylene variants, are all very susceptible to clinical creep when used in a lower extremity orthosis.

Clinical creep is also a drawback in spinal orthoses that are utilized to stabilize or straighten the spinal column. The optimal corrective forces of a spinal orthosis will decrease as clinical creep alters that shape of the orthosis and will decrease the corrective effectiveness of the orthosis.

As in orthoses, the sockets of prostheses are susceptible to shape change due to clinical creep when fabricated using thermoplastic materials, especially polypropylene. For these sockets, such creep has made pure thermoplastic materials and fabrication virtually unsuitable for definitive, long-term use in prosthesis.

Resins are used to create many types of products in the modern world, and in areas outside of orthoses and prostheses, in order to combat creep and confer additional strength; it is common to impregnate various types of fibers into, or along with, the thermoplastic resin. These fibers resist stretching along whatever angle that the fiber is aligned, but are generally ineffective at resisting compression along whatever angle that the fiber is aligned, and they are also generally ineffective at resisting bending perpendicular to whatever angle that the fiber is aligned. For example, substrate reinforcing fibers can be impregnated with uncured or unpolymerized thermoset resin either before or after the reinforcing fibers are placed in a product mold. These reinforcing fibers can confer additional dimensional stability and robustness to the final product. This is the general principle behind fiberglass, for example, which is used for a wide variety of different applications.

Various ways to incorporate fibers into thermoplastic resins are known. These methods include U.S. Pat. No. 3,523,149 (Hartmann), US Statutory Invention Registration H1162 (Yamamoto et al.), U.S. Pat. No. 6,054,022 (Helwig et al.), U.S. Pat. No. 4,478,771 (Schreiber), U.S. Pat. No. 5,071,608 (Smith et al.), U.S. Pat. No. 5,194,462 (Hirasaka et al.), and U.S. Pat. No. 5,741,744 (Fitchmun).

In one method, powdered thermoplastic resin is introduced into a woven, braided or a textile form of the continuous substrate fibers. The resulting material (often called a “prepreg” because the continuous substrate textile is pre-impregnated with the resin by the manufacturer, and then often shipped to the end user in the form of ready to use sheets or rolls) retains the textile characteristic of being “drapable”. This dry powdered prepreg can then be molded under high pressure and heated in a matched two-sided mold to consolidate the fibers and resin into a finished product.

Thermoplastic prepreg sheets that contain continuous fiber reinforcement in woven or braided form are commercially available. However due to the fact that the fibers are continuous and are present in a woven or braided form, these sheets are generally quite stiff. In order to fit these stiff sheets into complex molds, typically both matched (i.e. upper and lower mold surfaces) and sturdy molds (often made of metal) and high pressure and heat are required.

Unfortunately, the molding pressures that are used in prior art thermoplastic prepreg sheets are too high for use on the temporary plaster of Paris positive models used in the fabrication of orthoses and prostheses. Thus at present, the state of the art in the orthoses and prosthetic device field is generally to form the main body of the orthoses and prostheses out of the pure homogenous thermoplastic sheetstock, and to reserve any fiber reinforcement for certain strategic regions of the orthoses and prosthesis where it is absolutely essential, as in U.S. Pat. No. 6,146,349 (Rothschild et al.) and U.S. Pat. No. 5,312,669 (Bedard).

Although there is prior art with regards to using reinforcing fibers for orthoses and prosthesis devices, such as U.S. Pat. No. 6,146,344 (Bader), U.S. Pat. No. 5,624,386 (Tailor), U.S. Pat. No. 5,693,007 (Townsend), the results are still not fully satisfactory, particularly when durable orthoses and prosthesis devices must be constructed that must conform to complex body shapes, such as a three dimensional shape with a double horizon bend. As a result, the problem of clinical creep persists, and present day orthoses and prosthesis have both a limited use lifetime and a need for continual readjustment.

In U.S. patent application Ser. No. 12/901,549 (now U.S. Pat. No. 8,088,320) Bedard, also inventor of the present disclosure, proposed a material and method for reducing creep in orthotics and prosthetics. According to these methods, thermoplastic resin plies and fiber veil plies with a substantially porous structure composed of discontinuous fibers held together by small amounts of polymeric binder between the fiber contact points are consolidated under heat and pressure. This causes the resin to flow into the porous voids and the binder to dissociate from the contact points, producing a composite material with an embedded fiber veil remnant that retains the three dimensional structure of the original fiber veil. The various fibers can move freely with respect to one another at melt temperature, while the orientation of the fibers in the fiber veil remnant resists bending in all directions at lower temperatures. This composite material can then be vacuum thermoformed into at least one complex component of an orthotic device or prosthetic device, such as a complex component with a double horizon bend.

Since then, this approach has been found to be both clinically and commercially successful. In particular, the heavy gauge thermoplastic prepreg composite has proven to be successful in the production of orthoses (orthopedic braces or appliances) and prostheses (artificial limbs) and components thereof, usually by way of a vacuum thermoforming process. The application of the thermoplastic prepreg composite has been evaluated in a university setting with the subsequent publication of the results in the paper Effects of Materials, Reinforcement, and Heat Treatment on Thermoplastic Solid Ankle-Foot Orthosis Mechanical Properties: A Preliminary Study, Gao et al. 2013.

More specifically, U.S. patent application Ser. No. 12/901,549 (now U.S. Pat. No. 8,088,320) Taught ply stacks with various elements of thermoplastic sheetstock. The complete contents of U.S. patent application Ser. No. 12/901,549 are incorporated herein by reference.

The Ser. No. 12/901,549 disclosure taught specific examples, such as ply stacks with four elements of homogenous thermoplastic sheetstock in a thickness of 0.0625″ ( 1/16th″) and three elements of fiber veil. The application also taught sheetstock samples with three elements, where two plies were comprised of thermoplastic sheets and one ply element was the fiber veil. The Ser. No. 12/901,549 disclosure also taught that the thickness of the thermoplastic sheetstock can also vary with respect with the amount of flow required to produce a clinical product.

The Ser. No. 12/901,549 disclosure also taught thermoplastic composite materials that had the ability to undergo flow and/or draw during vacuum thermoforming so that it can be drawn from a flat sheet with a first wall thickness (often between a lower range of about 3/64 or 1/16 inches thick to an upper range of about ¾inches thick) into a three dimensional shape with a double horizon bend while maintaining a second wall thickness greater than a preset specification thickness. This disclosure also taught that this preset specification will be between about 75% to 125% of the original first wall thickness. The Ser. No. 12/901,549 disclosure also taught that this composite may typically comprise a plurality of thermoplastic material plies, and at least one internal fiber veil remnant ply. The Ser. No. 12/901,549 disclosure taught that this fiber veil remnant ply may be formed from discontinuous fibers, and it may have a morphology (three dimensional structure) derived from a heat or solvent dissociated original fiber veil composed of discontinuous fibers.

The Ser. No. 12/901,549 disclosure also taught a figure (Ser. No. 12/901,549 FIG. 4, here reproduced as FIG. 1) that illustrated a perspective of one embodiment of the Ser. No. 12/901,549 disclosure for a fiber stabilized and reinforced thermoplastic sheetstock (400) that is compatible with the process of heavy gauge, cut sheet, draped, encapsulation, and vacuum thermoforming. The Ser. No. 12/901,549 disclosure taught that sheetstock (400) can be comprised of layers of neat or homopolymer resin (402), (406) and layers of discontinuous fiber veil mat (404). The layers (402), (404), (406) can be consolidated under heat and pressure into a prepreg thermoplastic composite sheetstock (400) that can be reheated and molded into various articles with compound curves.

Ser. No. 12/901,549 also taught that the neat resin layers (402), (406) can be any linear or thermoplastic resin that is able to self-bond at melt temperature. The fiber layer (404) can consist of discontinuous fibers that can be comprised of carbon, fiberglass, aramind, or any reinforcing fibers such as organic fibers derived from coconut shells or even nanofibers. The neat resin layers (402), (406) can be of the same linear resin or compatible resins. As a decorative feature, resin layers (402) can be pigmented or colored in the same color or in multiple colors. As an example, one resin layer (402) could be colored blue, and one layer (402) could be colored blue. The outer layer (402) of a sample orthosis (500) could be in blue to appeal to a boy patient, while the outer layer (402) could be pink of an orthosis (500) to appeal to a girl patient.

Ser. No. 12/901,549 taught that the core resin layer (406) can have a thickness that is different than the outer resin layers (402). The disclosure also taught that a thicker core layer (406) will improve the mechanical characteristics of this sheetstock (400) by increasing the distance from the neutral axis of the prepreg composite to the reinforcing and stabilizing fiber layers (404). The outer layer (402) will also serve to protect a patient from the fiber layer (404) so there is no abrasion of the skin by the reinforcement fibers (404). The outer layers (402) will also serve to protect any article that is fabricated from this sheetstock (400) from abrasion of the reinforcing fibers (404).

Ser. No. 12/901,549 taught that this sheetstock (400) may consist of various layers of resin (402) and veil (404). In the simplest embodiment, the sheet stock (400) would consist of two outer layers (402) of neat resin and one fiber (404) core layer. A complex embodiment of the sheet stock (400) may consist of seven total layers in a preferred embodiment (400) with four layers of neat resin (402) and three layers of carbon veil (404). In the complex embodiment, one surface (402) of neat resin could consist of a resin that has a very low durometer. The low durometer could offer cushioning or dampening characteristics.

Cap Layers

Cap layers are relatively well known in the food packaging and electronics industry, and are often used for branding, and to provide air barriers for freshness preservation, and to protect laminated structures that hold sensitive electronic circuits. Examples of such art include Davis, U.S. Pat. Nos. 5,637,366; 6,033,514, and the like. Examples of cap layers include Dupont Pyralux® LF Coverlay, Bondply & Sheet Adhesive, Premier Material Concepts TPO, and the like. Cap layers are also discussed in the publication authored by Geoff Layhe entitled “Education and training in Electronic Design Realization Multilayer bonding—what's it all about?” Electronic Design Realization, www.edrcentre.org.uk (2002).

### BRIEF SUMMARY OF THE INVENTION

The present disclosure is based, in part, upon the insight that further refinements to prior art, as well as concepts previously disclosed in U.S. patent application Ser. No. 12/901,549 can be useful.

In particular, the invention is based, in part on the insight that although for clinical purposes it is often desirable to modify various properties of the surface layers of various multi-element thermoplastic materials, such surface modifications need not extend to any appreciable depth into the multi-element thermoplastic material. Thus extremely thin surface modifications may be desirable, because given that the overall thickness of the multi-element thermoplastic material may often have various thickness constraints, using a smaller amount of the total thickness for desired surface modifications thus frees up more thickness to achieve the required mechanical performance for the multi-element thermoplastic material.

The present invention is also based, in part, on the insight that cap layer technology, presently used in the packaging industry, but not in the field of providing thermoplastic materials for clinical purposes such as orthoses and prosthesis, may be utilized as a way to provide such extremely thin modifications to multi-element thermoplastic materials.

Thus the present disclosure focuses on materials and methods to provide extremely thin surface modifications to multi-element thermoplastic materials. As will be discussed, often these extremely thin surface modifications may be achieved by laminating extremely thin cap layers or membranes, with thicknesses often in the 2 to 12 mil (here a mil is 1/1000 inch) range, to one or more outer surfaces of the multi-element thermoplastic material.

Although, absent the cap layers, the inner multi-element thermoplastic materials may be constructed using the fiber veil remnant methods of previously discussed U.S. patent application Ser. No. 12/901,549, there is no requirement that these fiber veil remnant methods be used. In alternative embodiments, other methods to impart the necessary combination of mechanical properties to the multi-element thermoplastic materials may be used. Some of these other methods, which will also be discussed, include the use of short fiber filled thermoplastic piles. Indeed, the present disclosure's cap layer teaching can facilitate use of alternate short fiber filled thermoplastic materials. This is because the undesirable properties of short fiber filled thermoplastic materials, such as skin irritation, can be reduced or eliminated by the use of suitable extremely thin cap layers.

As will be discussed, use of cap layers can provide a systematic ability to configure many different types of enhanced thermoplastic sheetstock for orthoses and prosthesis. This sheetstock may be provided in various configurations with a wide cross section of physical characteristics. In particular, cap layers may enable a wide variety of different options including different processing options, structural options, clinical options, cosmetic options, anti-microbial options, and other options.

Thus in some embodiments, the invention may be viewed as a method of constructing a thermoplastic composite material with improved compatibility with human skin, as well as the resulting thermoplastic composite material, and an orthotic or prosthetic device comprising this thermoplastic material.

More specifically, the invention's methods, materials, and devices may comprise obtaining a clinical cap layer with at least one skin-side cap layer surface chosen for suitability for direct contact with human skin, and obtaining at least one resin layer comprising a polymeric resin. This at least one resin layer may have various properties, but it should be chosen for at least its ability to undergo flow and/or draw during vacuum thermoforming so that it can be drawn from a flat sheet with a first wall thickness into a three dimensional shape with a double horizon bend while maintaining a second wall thickness greater than a preset specification thickness. As will be discussed, these properties are important because they lend themselves to producing orthotic and prosthetic devices using vacuum thermoforming methods.

Here the clinical cap layer is consolidated with this at least one resin layer so as to provide a thermoplastic composite that has at least one skin-side cap layer surface which has been chosen for suitability for direct contact with human skin. At the same time, the skin-side cap layer is chosen to be thin enough so that the resulting thermoplastic composite continues to provide an ability to undergo flow and/or draw during vacuum thermoforming so that it can be drawn from a flat sheet with a first wall thickness into a three dimensional shape with a double horizon bend while maintaining a second wall thickness greater than a preset specification thickness. The net result is a composite material that, while still suitable for producing orthotic and prosthetic devices using vacuum thermoforming methods, produces improved orthotic and prosthetic devices that have improved compatibility for direct contact with human skin.

### BRIEF DESCRIPTION OF THE DRAWINGS

FIG. 1 shows an example of prior art multi-ply thermoplastic materials, such as those disclosed in U.S. patent application Ser. No. 12/901,549.

FIG. 2 shows an example of the present cap layer type multi-ply thermoplastic materials, which are the subject of the present disclosure.

FIG. 3 illustrates the typical process and apparatus utilized to fabricate a total contact Ankle Foot Orthosis (AFO) by heavy gauge, cut sheet, draped, encapsulation, and vacuum thermoforming process.

FIG. 4 is an illustration of a non-articulated Ankle Foot Orthosis (AFO).

FIG. 5 shows a complete endoskeleton prosthesis.

### DETAILED DESCRIPTION OF THE INVENTION

Previous art in the area of multi-ply thermoplastic sheetstock for orthoses and prostheses generally taught that each ply in the multi-ply structure was generally thick enough to also contribute to the mechanical strength of the overall composite material. By contrast, the present disclosure examines the consequences of dropping this earlier teaching in favor of using end cap material that, while contributing certain desirable properties to the multi-ply thermoplastic composite, is generally too thin to contribute to the mechanical strength of the composite to any significant extent.

When the main purpose of a given layer (ply) in a multi-ply composite is structural (i.e. mechanical strength), then such mechanical strength considerations can drive the selection of the polymer resin as well. This in turn places constraints on any other characteristics that a given layer (ply) may have, such as color and/or antimicrobial & antibacterial properties. Materials added to a layer (ply) to modulate its non-structural (e.g. color, anti-microbial properties, etc.) must be selected to also be compatible with that layer's polymer resin originally selected for that layer's structural purposes.

In the new teaching, the cap layer, which may only be 2-12 mils in thickness, but not restricted to that range, can be selected for the additive features independent of the outer or inner structural ply. Consequently the additive features of the cap layer can be optimized for physical characteristics as the additive cap layer completely independent of the design requirements of the structural ply.

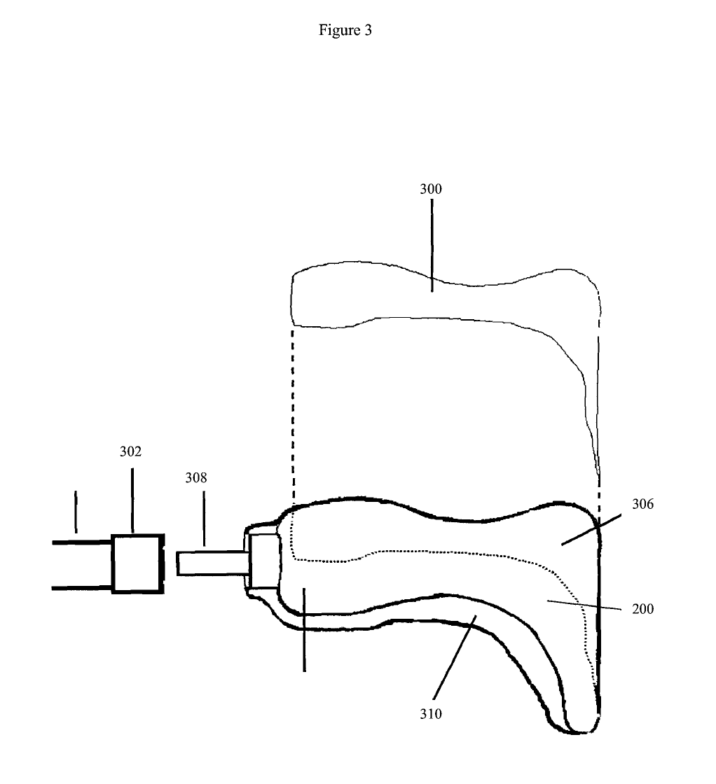

As previously discussed in U.S. patent application Ser. No. 12/901,549, one problem with thermoplastic materials for orthoses and prostheses is the problem of gradual stretching of the material, also called clinical creep. This problem can be reduced by embedding various fibers into the composite. FIG. 3 is a diagram showing how fibers (300), (302) in a composite material (304) can resist stretching forces along the direction of a fiber (306), but do not resist compression forces (308) and offer only small resistance to forces acting perpendicular to the direction of a fiber.

Although the stretching and deformation characteristics of thermoplastics can thus be substantially modified by filling the thermoplastic material with various types of fibers, when these fibers stick out of the thermoplastic material, they can cause problems. In particular, they can irritate the skin of patients, and also catch on clothing.

For this reason, prior art in the field teaches against the use of a fiber filled polymer resin on the inner or patient contact side of the composite, because the fiber may protrude from the surface and create a tissue abrasion issue with the patient's skin. The use of a fiber filled resin for the outer ply may also create friction issues with the patient's clothing. Thus prior art is not fully satisfactory in this regard.

Of course additional fiber free thermoplastic layers can be laminated on top of the fiber containing thermoplastic layers, and indeed this approach was proposed in Ser. No. 12/901,549, but here the problem is that the fiber free thermoplastic layers thus end up taking up an appreciable percentage of the overall thickness of the thermoplastic composite, and since the fiber free thermoplastic layers take up space, yet do little to solve clinical creep issues, this leaves less room in the composite for the fiber filled areas, thus producing an overall thermoplastic material that is less resistant to bending and stretching than would otherwise be the case.

By contrast, by moving to extremely thin cap layers, which may have little or no structural requirements, the requirement that the materials used in the cap layers for various properties (e.g. color, anti-microbial properties, etc.) also be compatible with the materials used for that layer's structural requirements can be eliminated or dropped. This in turn opens the door to a wider range of options.

In addition to providing desirable properties such as color and/or anti-microbial activity, cap materials, although perhaps contributing little or nothing to the ability of the thermoplastic composite to stand up to compression or deformation, can also be selected contribute some additional mechanical characteristics as well. From the standpoint of various clinical applications involving orthoses and prostheses, and as discussed in U.S. application Ser. No. 12/901,549 as well, small fibers protruding from the surface of the resin will irritate the patient's skin, and/or rub against the user's clothing causing undesired feature. However as Ser. No. 12/901,549 also discussed, such fibers may be needed to reduce bending and stretching, and also reduce clinical creep. Ser. No. 12/901,549 taught one way to cope with this issue, which was to use fiber veils, and surround the fiber veils with relatively thick non-fiber containing layers, thus providing ample space between the fiber veil remnants and the surface of the thermoplastic composite.

However according to the invention, extremely thin cap layers can also be chosen from materials selected to block protrusion of the fibers in the inner structural ply, as well as to optionally also and provide additional properties (additive features) as desired.

By contrast, according to one embodiment of the present disclosure, certain types of extremely thin cap layers can be placed on the exterior face of thermoplastic layers composed of even randomly oriented short fibers, such as fiber filled resin. These cap layers (clinical cap layers) can cover any protruding short fibers, and provide a desired smooth outer surface that doesn't irritate the user/patient's skin, and/or doesn't have undesired friction with the user's clothing.

Thus in one aspect, use of extremely thin, and essentially non-structural cap layers enable the use of a much broader variety of thermoplastic fiber fillers. In particular, use of a wider variety of different short fibers for the now inner layers (plies) becomes feasible, which in turn can lead to improved thermoplastics with the same overall thickness or even less overall thickness, but also less clinical creep, relative to prior art in this field.

As a result, the invention enables use of both of more fibers, and a broader variety of fiber types.

For example, cap layers make it more feasible to use glass fibers as thermoplastic resin fillers. For example, using polypropylene as an example of thermoplastic resin, and glass fibers as an example of the fibers, use of relatively short (typically 4 mm length) glass can be contained within the standard dimension of a raw resin pellet, and the percentage by weight of the glass fibers in the pellet can range from about 10-40%.

Such thermoplastics with, for example, 20% glass fiber content can result in tensile strength improvements from 4.2K PSI tensile strength (no glass fiber) to 10.2K PSI (20% glass fiber).

In practice, the percentage of fiber content can be varied according to needs and requirements. Here compounding is relatively simple. The raw polymer resin itself is available in powder, flake and pellet form. Typical commercial resin pellets have diameters that typically range from about 3.2 to 12.7 mm, and generally the length of the glass fibers in these commercially available thermoplastic-resin glass-fiber pellets vary according to the diameter of the pellet.

Other types of fiber fillers may also be used. In regards to nano fiber and nano tubes, and even carbon nanotubes, these fibers also offer the ability to change the resin characteristics.

As previously discussed in FIG. 3, the standard form of a composite receives its benefit from the combined interaction of a polymer resin with a reinforcement fiber, not unlike adding straw to mud for adobe construction. Nano fibers also reinforce, although on a smaller scale. Thus in some embodiments, carbon nano fibers can interact with the molecular elements of the resin.

Consequently in some embodiments, the structural resin plies may comprise resin that has been modified by various nano fibers, such as carbon nanotubes and the like. In turn, the nano-fiber loaded resin plies, along with the cap layer, and optionally further incorporating larger fiber veil remnant fibers as per Ser. No. 12/901,549, can help create a range of products with different physical characteristics as desired.

Thus in some embodiments, the present disclosure teaches a new type of orthotic and prosthetic thermoplastic composite that combines the benefits of cap layers with the structural benefits of interior thermoplastic resin plies that have been reinforced with a wide variety of fiber type materials, including nothing (e.g. just pure thermoplastic material), carbon nanotubes or other types of carbon nanofibers, fiber veils, glass fibers, and other types of fibers as desired.

Various types of cap layers may be used, these include:
cap layer without additives
cap layer with additive (antimicrobial)
cap layer with decorative feature color and/or image
cap layer with durometer chosen for clinical compatibility with the patient's skin
cap layer with adhesion (suspension element)—here, for example, the cap layer can have an exposed surface that is tacky or gummy to the touch even after the cap layer has been adhered or laminated to the thermoplastic composite structure.
cap layer with conductivity—these layers may be configured with various types of conducting materials and configured for various clinical and non-clinical purposes, including clinical applications such as picking up electromyographic activity from muscle activity, and/or adding a thermal heating capability to the patient cap layer; or for cosmetic purposes such as absorption of at least certain electromagnetic waves as an isolating cap layer.

The interior of the multi-ply thermoplastic composite structure can contain one or more various structural plies. These can include
structural ply neat resin (e.g. no added fibers)
structural ply with fiber filled resin
structural ply with nanofibers (e.g. carbon nanotubes and the like)

Additionally, as per Ser. No. 12/901,549, the inner structure of the thermoplastic composite may also contain fiber veil remnant, as well as other fibers that vary according to:
Fiber type
Fiber content (weight)
Fiber combination (glass & carbon fiber)
Fiber organic (coconut fibers, hemp fibers)

Thus, as previously discussed, in some embodiments the invention may be a method of constructing a thermoplastic composite material with improved compatibility with human skin. This method will generally comprise obtaining a clinical cap layer with at least one skin-side cap layer surface chosen for suitability for direct contact with human skin, as well as well as obtaining at least one resin layer comprising polymeric resin.

In a preferred embodiment, the clinical cap layer may be relatively thin, with a thickness roughly between 2/1000 and 12/1000 of an inch. This clinical cap layer can be chosen from a material selected for its abilities for at least one of reducing skin irritation, low friction with skin, cushioning (durometer) value, hypoallergenic properties, or antimicrobial activity.

In some embodiments, the clinical cap layer can be further selected according to color, UV resistance, cosmetic appearance, or ability to improve thermoplastic material resistance to sag during a melt phase of a subsequent vacuum forming process.

In some embodiments, the clinical cap layer further comprises at least one of a polyimide containing film, thermoplastic poly olefin containing film, biaxially-oriented polypropylene film, or other polypropylene containing film. However other types of materials may also be used. This clinical cap layer can be consolidated with the thermoplastic composite by any of a co-extrusion, heat melt lamination, heat adhesive lamination process, or other adhesive lamination process.

The at least one resin layer is generally chosen for at least an ability to undergo flow and/or draw during vacuum thermoforming, so that it can be drawn from a flat sheet with a first wall thickness into a three dimensional shape with a double horizon bend while maintaining a second wall thickness greater than a preset specification thickness.

In some embodiments, this at least one resin layer further comprises at least some protruding fibers, and the clinical cap layer chosen at least in part for its ability to prevent these protruding fibers from direct contact with the human skin of the patient who subsequently is wearing or will wear an orthotic or prosthetic device formed from this material.

In some embodiments, this at least one resin layer may further comprise at least one structural ply layer with fiber fillers, and/or structural plies without fiber fillers. If used, these fiber fillers may comprise various types of fibers such as carbon fibers, glass fibers, graphite fibers, polymeric fibers, aramide fibers, plant fibers, carbon nanofibers, and carbon nanotubes.

In some cases, at least some of the structural plies can also comprise either a mixture of both resin and fibers, or a carbon fiber veil or carbon fiber veil reminant ply, as per the methods of U.S. patent application Ser. No. 12/901,549 (now U.S. Pat. No. 8,088,320).

The at least one resin layer can comprise various types of materials, including materials selected from the group consisting of Acrylonitrile Butadiene Styrene (ABS) resin, acrylic/PVC thermoplastic sheets (Kydex), polycarbonate resin (Lexan), co-polyester sheets (VIVAK), thermoplastic elastomer (TPE) resins, polyvinyl chloride (PVC) resins, polyolefin resins, polypropylene resins, isotatic polypropylene resins, polystyrene, homopolymer polypropylene, co-polymer polypropylene, polyethylene, and other thermoplastic materials.

The method further comprises consolidating this clinical cap layer with this least one resin layer so as to provide a thermoplastic composite with at least one skin-side cap layer surface chosen for suitability for direct contact with human skin. Here at least one important constraint is that the resulting thermoplastic composite should also provide an ability to undergo flow and/or draw during vacuum thermoforming. This allows the thermoplastic composite to be drawn from a flat sheet with a first wall thickness into a three dimensional shape with a double horizon bend while maintaining a second wall thickness greater than a preset specification thickness. This in turn makes the thermoplastic composite suitable for producing improved orthotic and prosthetic devices using standard orthotic and prosthetic device production methods.

In a preferred embodiment, the thermoplastic composite will have a first wall thickness about between 3/64 inches and ¾inches, and have a preset specification thickness between about 75% to 125% of this first wall thickness.

Although thermoplastic composites with only one clinical cap layer (e.g. a skin side layer) are contemplated according to the invention, in some embodiments both sides of the thermoplastic composite will have cap layers. Often the cap layer opposite the skin side clinical cap layer will not be in direct contact with the human patient's skin, but this cap layer opposite to the skin side layer will often be exposed to the outside world, and outside observers. Thus the invention further contemplates thermoplastic composites with two cap layers: a skin side layer, and an opposite cap layer that may have more of a cosmetic purpose. This opposite cap layer that may have more of a cosmetic appearance will be termed a “cosmetic cap layer”.

Thus some embodiments of the invention will further comprise obtaining a cosmetic cap layer, and further consolidating this cosmetic cap layer on a side of the at least one resin layer that is opposite to the clinical cap layer. Generally this cosmetic cap layer is not intended for direct contact with human skin. Instead, the cosmetic cap layer may often be chosen for other properties, such as its cosmetic properties, UV resistance properties or ability to improve thermoplastic material resistance to sag during a melt phase of a subsequent vacuum forming process. This cosmetic cap layer may be formed from a variety of different materials, including polyimide containing films, thermoplastic poly olefin containing films, biaxially-oriented polypropylene films, or other polypropylene containing films.

This cosmetic cap layer will also be relatively thin, typically with a thickness between 2/1000 and 12/1000 of an inch. Like the clinical cap layer, the cosmetic cap layer can be consolidated with the at least one resin layer by any of a co-extrusion, heat melt lamination, heat adhesive lamination process, or other adhesive lamination process.

Once the complete thermoplastic material is produced, it can then be used to further construct an orthotic device or a prosthetic device for a patient using said composite material. This can be, for example, an orthotic device or a prosthetic device for a patient, which is formed from this thermoplastic composite material with improved compatibility with human skin.

As previously discussed, this orthotic or prosthetic device will generally comprise

1: A clinical cap layer with at least one skin-side cap layer surface chosen for suitability for direct contact with human skin.
2: At least one resin layer comprising polymeric resin. As previously discussed, this at least one resin layer will generally be chosen for at least some ability to undergo flow and/or draw during vacuum thermoforming so that the at least one resin layer can be drawn from a flat sheet with a first wall thickness into a three dimensional shape with a double horizon bend while maintaining a second wall thickness greater than a preset specification thickness.
3: This at least one resin layer is consolidated with a clinical cap layer so as to provide a thermoplastic composite with at least one skin-side cap layer surface that has been chosen for suitability for direct contact with human skin.
4: At the same time, the thermoplastic composite is still such that it provides an ability to undergo flow and/or draw during vacuum thermoforming so that it can be drawn from a flat sheet with a first wall thickness into a three dimensional shape with a double horizon bend while maintaining a second wall thickness greater than a preset specification thickness, as discussed previously.

As previously discussed, generally the thermoplastic composite used in this device will have a clinical cap layer with a thickness between 2/1000 and 12/1000 of an inch, and this clinical cap layer will be made from (comprise) a material selected for its abilities to do at least one of reducing skin irritation, low friction with skin, cushioning (durometer) value, hypoallergenic properties, or antimicrobial activity. As previously discussed, this clinical cap layer may be made from various types of material including polyimide containing films, thermoplastic poly olefin containing films, biaxially-oriented polypropylene films, other polypropylene containing films, and the like.

### Examples

Some specific examples of various types of thermoplastic composite sheetstocks, suitable for orthoses and prostheses, include, in order from simplest to most complex:

1: A three layer composite sheet comprising:
a) An inner patient contact cap layer (clinical cap layer) offering antimicrobial & antibacterial properties;
b) A core of fiber filled polymer resin (but no fiber veil);
c) And an outer cosmetic cap layer offering color option.
   2: A three layer sheet comprising:
a) An inner patient contact cap layer (clinical cap layer) offering lower durometer for tissue cushioning;
b) A core of resin reinforced with various types of nanofibers, but no fiber veil:
c) An outer cosmetic cap layer offering a metallic cosmetic option.
   3) An eight layer sheet comprising:
a) An inner lower friction patient contact cap layer (clinical cap layer) offering reduced tissue shear characteristics;
b) An inner structural polymer ply with nano carbon tube enhanced resin;
c) A carbon fiber veil ply;
d) A center core ply of nanofiber enhanced resin;
e) A carbon fiber veil ply;
f) An outer structural core of carbon nanofiber enhanced resin;
g) A carbon fiber veil ply;
h) An outer cosmetic cap layer offering a decorative design such as a camouflage coloring.

Thus as can be seen, the invention allows a much greater variety of thermoplastic materials to be systematically engineered for orthoses and prostheses, as well as other purposes than was previously possible.

Further discussion and alternative embodiments:

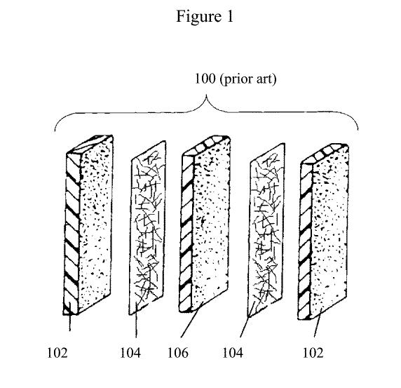

As shown in FIG. 1, in some embodiments, application Ser. No. 12/901,549 taught a plurality of thermoplastic plies and one original fiber veil, such as two fiber veils with three thermoplastic plies. The outer surface thermoplastic plies provided one (inner) surface that was in potential contact with the patient's skin and another (outer) surface that was visible to the outside world when the patent was wearing the orthoses or prostheses (The middle or core thermoplastic ply(s) of homogenous thermoplastic resin served as a means to keep the fiber veil away from the neutral axis of the prepreg composite which provided a mechanical benefit to the composite structure.)

As previously discussed, one embodiment of the new multi-element sheetstock is the use of a fiber filled thermoplastic ply for the middle core ply. Fiber filled thermoplastic resins are commonly available for injection molding. It is uncommon to use a fiber filled thermoplastic resin in a sheet form for vacuum thermoforming as the short fibers may protrude from the surface of the material in the finish product. The protrusion of the short reinforcement from the inner clinical surface may create tissue trauma when in contact with the patient's skin. The protrusion of the short fibers in the outer thermoplastic ply may cause undue wear of the patient's clothing as clinical devices are mainly worn under clothing. Another disadvantage of using a fiber filled resin in sheetstock in a vacuum forming application is the possible lack of sag strength when at melt temperature. Lack of sag strength increases the difficulty of physically manipulating the sheetstock at melt temperature onto a mold suitable for vacuum thermoforming.

The advantage of using a fiber filled ply as the core in a multi-element sheetstock is the isolation of the core from the surface of the sheetstock. The short fibers are thus prevented from protruding from the surface of the finished product thereby preventing any opportunity to create friction when in contact with the patient's skin. The use of a short fiber filled thermoplastic core will also isolate the fibers from the outer surface of the clinical device and thus avoid a high friction surface inducing undue wear on the patient's clothing. The added benefit of using a fiber filled resin for the core ply in the multi-element sheetstock is the gain in structural integrity of the overall sheet through the use of a commonly available fiber filled resin without the risk of tissue trauma to the patient.

Also as previously discussed, another preferred embodiment of the new multi-element sheetstock is the use of either a clinical cap layer or a cosmetic cap layer on the outer thermoplastic resin plies. The cap layer is a thin film of material on the surface of thermoplastic ply. The cap film layer can be a cosmetic cap layer that can serve as a decorative function such as offering a color or image choice on the visible surface of the clinical device. A UV resistant cosmetic cap layer on the visible surface of the multi-element sheetstock, when incorporated into a clinical device, can also offer UV protection to the underlying outer thermoplastic resin ply. The cap film material, when used as a clinical cap layer, can serve a clinical function when on the inner patient contact side of the multi-element sheetstock. The clinical function of the inner cap ply (clinical cap layer) can offer a hypoallergenic benefit, an antimicrobial benefit, a suspension element to help maintain attachment of the clinical device to the patient, a cushioning element to decrease tissue trauma from pressure, and a friction reduction element to decrease shear trauma to the patient's skin. Both types of cap layer can be added to the thermoplastic ply by co-extrusion, via heat melt lamination, or heat adhesive lamination before incorporation into the enhanced multi-element sheetstock.

In another embodiment of the new multi-element sheetstock is that the outer thermoplastic plies of the multi-element sheetstock can utilize a fiber filled thermoplastic resin. The cap layer when used on the inner clinical surface as a clinical cap layer, or the outer visible surface of the clinical device as a cosmetic cap layer, will block the protrusion of the short fibers from protruding from the surface of the multi-element sheetstock. The cap layer(s) can also offer the benefit of additional/improved sag strength during the melt phase of the vacuum forming process.

In a simplified example of this embodiment of the new multi-element sheetstock, the fiber veil ply may be eliminated. In this rendition, the inner core ply consists of a short fiber filled thermoplastic resin. The core ply is covered with an inner (clinical) and outer (cosmetic) cap layer. The exposed cap layers can provide additional sag strength for the melt phase thermoforming process. The inner clinical surface cap layer (clinical cap layer) can provide the previous indicated benefits of hypoallergenic, antimicrobial, suspension and shear reducing elements. The outer visible cap layer (cosmetic cap layer) will provide cosmetic elements. The inner and outer cap layers will prevent protrusion of the short fibers from the surface when fabricated into a clinical device.

A more complex embodiment of the multi-element sheetstock in this example may incorporate seven layered elements. The outer cosmetic cap layer that is visible to the patient in a clinical device can serve as a decorative element, which may increase patient compliance in wearing the device for the intended medical benefit. The visible cosmetic cap layer may cover the outer thermoplastic ply that has the option of being fiber filled to offer an increase in structural integrity to the device. The outer cosmetic cap layer and outer thermoplastic ply would then cover the first fiber veil ply, and offer the benefit of strength to the composite material.

Some advantages over the material previously described in the Ser. No. 12/901,549 (now U.S. Pat. No. 8,088,320) is that new material describe in this present disclosure provides a new multi-element material that has can use glass filled thermoplastic plies to provide higher levels of structural integrity in the finished clinical device, while at the same time avoiding the problems of glass fibers, such as irritated patient skin.

In some embodiments, the core ply layer of the multi-element sheetstock has the option of being a fiber filled thermoplastic resin. The fiber filled core thermoplastic layer may add structural integrity to the device. The core thermoplastic layer can then be integrated with the inner fiber veil, which is then integrated with the inner thermoplastic resin layer. The inner thermoplastic resin layer also has the option of being covered by a cap layer (clinical cap layer) on the patient contact surface of the clinical device.

In this more complex embodiment, the inner cap film (clinical cap layer) on the inner clinical surface can offer a hypoallergenic benefit, an antimicrobial benefit, a suspension element to help maintain attachment of the clinical device to the patient, a cushioning element to decrease tissue trauma from pressure, a sealing element for enhancement of vacuum suspension and a friction reduction element to decrease shear trauma to the patient's skin. The clinical cap layer serves to cover the inner thermoplastic resin layer, which also has the option of being a fiber filled thermoplastic resin.

In a more sophisticated embodiment of the multi-element sheetstock, a plurality of thermoplastic plies, plurality of fiber veil elements, a plurality of cap layers can offer clinical, cosmetic, structural and process benefits that in the production of orthoses (orthopedic braces or appliances) and prostheses (artificial limbs) and components thereof. As previously discussed, usually such orthoses and prostheses can be formed by way of a vacuum forming process, and this can offer improved benefits over prior art.

Alternatively, in a more simplified embodiment of the invention, the multi-element sheetstock can be formed using only a single fiber veil elements, or even no fiber veil elements, and simply provide an inner (clinical cap layer) and outer (cosmetic) cap layer on a core ply of polymer resin consisting of fiber filled thermoplastic.

The benefit of this simplified multi-element sheetstock is that the inner (clinical) cap layer can offer an inner clinical surface with at least one of the previously described benefits such as hypoallergenic benefits and antimicrobial benefit. Additionally, the clinical cap layer can also provide optional additional benefits such as at least one of a suspension element to help maintain attachment of the clinical device to the patient, a cushioning element to decrease tissue trauma from pressure, and a friction reduction element to decrease shear trauma to the patient's skin.

As previously discussed, the outer cosmetic cap layer would be visible to the patient in the clinical device, and thus can serve as a decorative element. This in turn may increase patient compliance in wearing the device for the intended medical benefit, particularly for child patients.

As previously discussed, the outer cosmetic cap layer can also offer additional benefits, such as UV protection to the inner thermoplastic fiber filled polymer. The cosmetic cap layer can also add sag strength, which will be very useful during the melt temperature physical manipulation of the sheet during prosthetic and orthotic device vacuum forming steps. Additionally, as previously described, the cosmetic cap layer can also help prevent protrusion of the fibers from the inner thermoplastic resin to the outer surface.

Another embodiment of the new multi-element sheetstock is the addition of molecular additives to the thermoplastic resin layer. According to prior art methods, the use of various macroscopic fibers, such as fiber veils and short fibers, to produce a fiber filled thermoplastic resin layer is known to be useful, because such macroscopic fibers can serve as a means to modify the physical characteristics of the multi-element sheetstock (over the use of a heavy gauge homogenous or neat thermoplastic resin sheetstock).

By contrast, use of molecular additives such as carbon nano fibers and carbon nano tubes to thermoplastic resin to improve the properties of multi-element sheetstock is comparatively unexplored. In another embodiment of the invention, the physical characteristics of the thermoplastic resin material can be favorably modified on a molecular level by adding various nano elements, such as carbon nano fibers, and carbon nanotubes, to either the cap film layers or the underlying thermoplastic resin material itself. The inclusion such nano elements (atomic scale structural elements) embodies another one of the various options that are available to the overall multi-element sheetstock system according to the invention.

As previously discussed, the invention further provides a clinical device, such as an orthosis or prosthesis, reinforced with an end cap reinforced discontinuous fiber stabilized thermoplastic sheetstock.

If used with fiber veil type materials, the underlying thermoplastic resin sheetstock may be fabricated with a veil fiber weight that is lower than this sheetstock that has a higher or greater volume or weight of discontinuous fibers. Additionally, a reinforcement coupon material with greater stiffness than the main host sheetstock may also be used for fabricating various types of clinical devices. Here the methods previously discussed in Ser. No. 12/901,549 (now U.S. Pat. No. 8,088,320), the contents of which are incorporated herein by reference in their entirety, may be used.

### Additional Discussion:

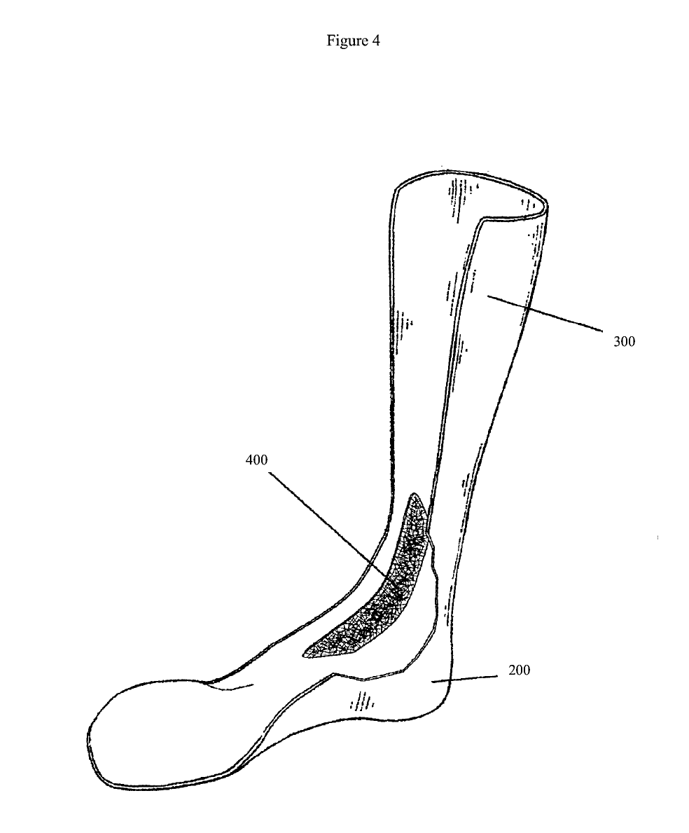

FIG. 1, originally taken from FIG. 4 of patent application Ser. No. 12/901,549 (now U.S. Pat. No. 8,088,320), illustrates the prior art in this area. According to prior art, a fiber stabilized and reinforced thermoplastic sheetstock (100) that is compatible with the process of heavy gauge, cut sheet, draped, encapsulation, and vacuum thermoforming, but without end caps, is provided. The sheetstock (100) was comprised of layers of neat or homopolymer resin (102), (106) and layers of discontinuous fiber veil mat (104). The layers (102), (104), (106) were consolidated under heat and pressure into a prepreg thermoplastic composite sheetstock (100) that can be reheated and molded into various articles with compound curves.

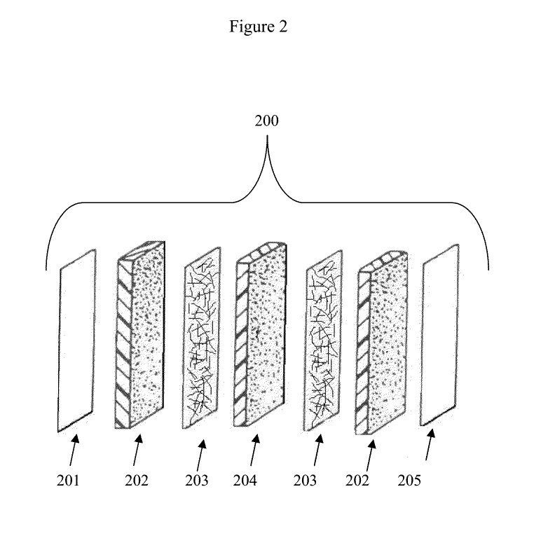

FIG. 2 illustrates a fragmentary perspective of one embodiment of this invention for an end cap equipped, fiber stabilized and reinforced, multi-element thermoplastic sheetstock (200) that is also compatible with the process of heavy gauge, cut sheet, draped, encapsulation, vacuum thermoforming. The sheetstock is comprised of cap layers (both clinical cap layers and cosmetic cap layers) of neat or homopolymer resin (201), (205) and plies of neat or homopolymer resin (202), (204). The multi-element sheetstock may optionally also be comprised of layers of discontinuous fiber veil mat (203), but such fiber veil mats are not a required component of the invention.

The optional fiber veil mat (203) may or may not be incorporated into the final sheetstock depending on the required physical characteristics within the span of products offered from the multi-element system of sheetstock. The layers (201), (202), (203), (204), and (205) are consolidated under heat and pressure into a prepreg thermoplastic composite sheetstock that can be reheated and molded into various articles with compound curves.

As previously discussed, cap layers (201), (205) can, in some embodiments, be made of a material can self-bond at melt temperature or bond to the resin layer (202), (205) with a heat activated adhesive. If the cap layers (201), (205) contain a resin material, this resin material may be a neat or homopolymer resin.

The cap layer material (201), (205) can further be composed of a material contains nano molecular elements. The cap layer(s) can be added to the thermoplastic ply by co-extrusion or via a lamination process before incorporation into the enhanced multi-element sheetstock. As an example the cap layer (201) can be a clinical cap layer that serves as the inner clinical contact surface. The clinical function of the inner clinical cap layer (201) can offer a hypoallergenic benefit, an antimicrobial benefit, a suspension element to help maintain attachment of the clinical device to the patient, a cushioning element to decrease tissue trauma from pressure, and a friction reduction element to decrease shear trauma to the patient's skin. The cap layer (201) can also block protrusion of short fibers when those fibers are used as an element in a fiber filled resin layer (102).

The outer cosmetic cap layer (205) can also serve as the outer visible surface of the clinical device. As previously discussed, the outer cosmetic cap layer (205) can serve as a decorative function such as offering a color or image choice on the visible surface of the clinical device. The cosmetic cap layer (205) on the visible surface of the multi-element sheetstock, when incorporated into a clinical device, can also offer UV protection to the underlying outer thermoplastic resin ply. The cap layer (205) can offer a mat or dull finish or a gloss or shiny surface. The cap layer (201), (205) when used in combination as an inner clinical contact surface (201) and as the outer cosmetic surface (205) can offer a processing benefit. The combination of the inner and outer cap layer (201) & (205) offers sag strength to the consolidated multi-element sheetstock when at melt temperature during the vacuum thermoforming process.

The neat resin plies (202), (204) can be of the same linear resin or compatible resins. The (202), (204) resin plies can also be comprised of resin filled with short reinforcement fibers. The (202), (204) resin plies can be comprised of resin that contains molecular nano elements. As an example, the (202), (204) resin plies may contain resin with molecular nano elements such as nano carbon fibers or carbon nano tubes. The (202), (204) resin plies can also contain fibers that are organic in nature such as fibers derived from coconut fibers or hemp fibers. The core resin ply (204) can have a thickness that is different than the outer core resin plies (202). A thicker core ply (204) will improve the mechanical characteristics of this multi-element sheetstock by increasing the distance from the neutral axis of the prepreg composite to the reinforcing and stabilizing fiber layers (202).

The optional fiber layer (203), which optionally may be a fiber veil layer, consists of discontinuous fibers that can be comprised of carbon, fiberglass, aramind, or any reinforcing fibers such as organic fibers derived from coconut shells or hemp fiber or fibers enhanced with nano technology. The discontinuous fibers can be comprised of a mixture of fiber types.

In some embodiments, a commercial veil of discontinuous fibers can be used in sheet form as a textile substrate, and placed in a ply stack combining extruded resin ply (202), (204) and cap layers (201), (205). This embodiment uses a fiber veil with a binder that demonstrates a disassociation when exposed to the melt temperature range of this resin ply (202), (204). Once heat and pressure are applied to the multi-element stack, the thermoplastic resin at melt temperature flows into the porous veil and completely encapsulates the discontinuous fibers in the veil. The heat and pressure consolidation of the porous veil with the thermoplastic resin multi-element components produces a prepreg thermoplastic composite laminate sheetstock that is suitable for heavy gauge vacuum thermoforming.

A simple perspective of this embodiment of the multi-element sheetstock comprises a clinical cap layer (201) as the inner clinical surface, a single resin core layer (204) of resin filled with short reinforcement fibers, and a cosmetic cap layer (205) as the visible cosmetic surface. The combination of inner clinical cap layer (201) and outer cosmetic cap layer (205) can optionally also serve to add additional sag resistance strength to the sheetstock during melt temperature manipulation in the vacuum thermoforming process. The elimination of the fiber veil ply (203) allows for a multi-element sheetstock that has lower strength characteristics that a multi-element sheetstock example that utilizes the fiber ply (203), and can be used for these purposes when desired.

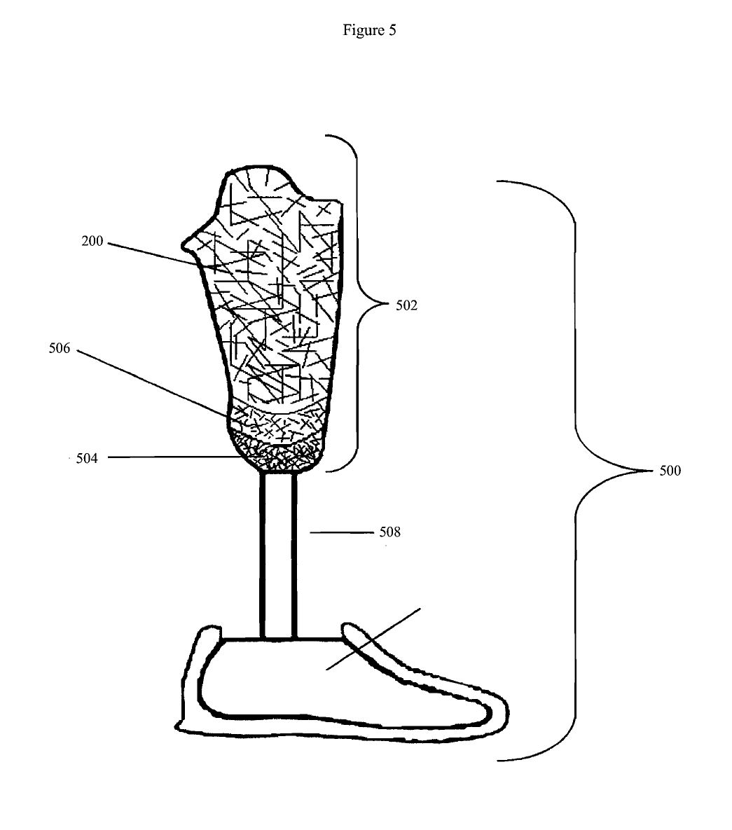

FIG. 3, originally taken from FIG. 5 of patent application Ser. No. 12/901,549 (now U.S. Pat. No. 8,088,320), illustrates the typical process and apparatus utilized to fabricate a total contact Ankle Foot Orthosis (AFO) (300) by heavy gauge, cut sheet, draped, encapsulation, and vacuum thermoforming. Vacuum is applied through a manifold (302) to the undersurface of the preferred, end cap equipped sheet stock (200) which is the previously discussed thermoplastic material in sheet stock form. This will be referred to in FIGS. 3-6 as the “preferred sheetstock”. The preferred sheetstock (200) has been heated to melt temperature and formed over a positive model (306) of the patient's lower limb. A pipe mandrel (308) has been embedded into the plaster of Paris positive model (306) to enable the positive model (306) to be secured into the vacuum manifold (302). Once the preferred sheetstock (200) is molded over the positive model (306), a vacuum force is applied through the vacuum mandrel (302).

The preferred end cap equipped sheetstock (200), which in some configurations (depending on choice of end cap material) may be self-bonding at melt temperature, is sealed (by either self-bonding, other adhesive, or other fastening process) along the bottom edge of the positive model (306) which forms a double thickness (310) of the preferred sheet stock (200). Once the preferred sheetstock (200) has cooled and stabilized, it is trimmed from the positive model (306), and provides for a rough ankle foot orthosis (AFO) (300) that is then edge finished with various power tools.

FIG. 4 is an illustration of a non-articulated Ankle Foot Orthosis (AFO) (300). The anterior and posterior stiffness of the AFO (300), which has been vacuum thermoformed from the preferred sheetstock (200), can be enhanced through the use of an inner surface reinforcement (400). The inner surface reinforcement (400) can consist of an extra layer of the preferred sheetstock (200) or other material. The inner surface reinforcement (400) can be of any shape that will enhance the stiffness of the device in any inner surface area of the device. The inner surface reinforcement can contain fiber layers that are the same weight as the host preferred sheetstock (200), or other types of materials, or the inner surface reinforcement (400) can consist of a preferred sheetstock (200) that has fiber layers of a higher fiber density. In this manner, extra reinforcement is limited to high stress areas of the device, and thus minimizing the overall weigh of the device by not requiring a heavier weight fiber layer to be used for the whole device.

The inner surface reinforcement (400) is applied during the vacuum forming process. The inner surface reinforcement (400) may be of the same or different neat resin as the preferred sheetstock (200), and may bond at melt temperature when host preferred sheetstock (200) and the inner surface reinforcement (400) are all heated to melt temperature in preparation of molding during the vacuum thermoforming process. Depending on the end cap material used in the preferred sheetstock, either no adhesive or some adhesive may be required for the bond between the inner surface reinforcement (400) and the host preferred sheetstock (200).

In FIG. 5, a complete endoskeleton prosthesis (here an enodskeletal prostheses means that a pylon is used as the structural member for weight support between the socket and the prosthetic foot. By contrast an exoskeletal prosthesis relies on fiberglass molded over a wood core for structural integrity) (500) is illustrated. The prosthesis (500) incorporates a prosthetic socket (502) that is vacuum thermoformed from another embodiment of the preferred sheetstock (200). The preferred sheetstock (200) in this embodiment, in addition to the previously discussed end cap material, may optionally also incorporate central layers of fiber veil that are stacked with several inner fiber layers that are circular and central to the preferred sheetstock (200). The density of the fiber layers may be heaviest in the smaller diameter layer (504). Another layer of fiber material (506) is larger than the heavier density smaller fiber material (504) and the larger fiber circular layer (506) is of a lighter density or weight than the smaller fiber material (504). The larger (506) fiber material diameter layer and the smaller diameter fiber material layer (504) may, in some embodiments, be incorporated into the preferred sheetstock (200) as integral layers.

Once the preferred sheetstock (200) is heated to melt temperature and formed over an appropriate plaster of Paris positive model of the patient's residual limb, the circular central layers of the smaller (504) and larger (506) diameter fiber material segments may be vacuum formed over appropriate prosthetic componentry that allows a connection between the prosthetic socket (502) and the pylon (508) of the prosthesis. The centralized area of increased fiber material density in the smaller (504) and larger (506) diameter fiber materials will increase the strength of the high stress connection between the pylon (508) and the prosthetic socket (502).

The proximal area of the prosthetic socket (502) may contain a lower fiber density of preferred sheetstock (200), thus offering some flexibility to the socket to improve comfort to the patient. The proximal area of the prosthetic socket (502), with the lower fiber density may also allow better post-delivery comfort adjustments to the socket through the use of thermal heating and recontouring of the walls of the prosthetic socket (502).

Note that although the cap layer enhanced thermoplastic materials described herein are highly useful for orthoses and prostheses, this is not the only area where such materials are useful. In other embodiments, such materials may also be used for other industrial purposes, such as to convey extra UV protection or the other properties described herein to thermoplastic rolls and sheetstock for any application where such properties are desired.

Kydex is a registered trademark of Kydex, LLC. Lexan is a registered trademark for SABIC Innovative Plastics. Vivak is a registered trademark of Sheffield Plastics, Inc.

1. A thermoplastic composite material with improved compatibility with human skin, said material comprising:
a clinical cap layer with at least one skin-side cap layer surface chosen for suitability for direct contact with human skin;
at least one resin layer comprising polymeric resin, said at least one resin layer chosen for at least some ability to undergo flow and/or draw during vacuum thermoforming so that it can be drawn from a flat sheet with a first wall thickness into a three dimensional shape with a double horizon bend while maintaining a second wall thickness greater than a preset specification thickness;
said at least one resin layer being consolidated with said clinical cap layer so as to provide a thermoplastic composite with at least one skin-side cap layer surface chosen for suitability for direct contact with human skin, while also providing an ability to undergo flow and/or draw during vacuum thermoforming so that it can be drawn from a flat sheet with a first wall thickness into a three dimensional shape with a double horizon bend while maintaining a second wall thickness greater than a preset specification thickness.

2. The material of claim 1, wherein said clinical cap layer has a thickness between 2/1000 and 12/1000 of an inch, and wherein said clinical cap layer is consolidated with said thermoplastic composite by any of a co-extrusion, heat melt lamination, heat adhesive lamination process, or other adhesive lamination process.

3. The material of claim 1, wherein said clinical cap layer is chosen from a material selected for its abilities for at least one of reducing skin irritation, low friction with skin, cushioning (durometer) value, hypoallergenic properties, or antimicrobial activity.

4. The material of claim 3, wherein said clinical cap layer is further selected according to color, UV resistance, cosmetic appearance, conductivity, or ability to improve thermoplastic material resistance to sag during a melt phase of a subsequent vacuum forming process;
and wherein said clinical cap layer further comprises at least one of a polyimide containing film, thermoplastic poly olefin containing film, biaxially-oriented polypropylene film, or other polypropylene containing film.

5. The material of claim 3, wherein said at least one resin layer further comprises at least some protruding fibers, and said clinical cap layer is chosen for its ability to prevent said protruding fibers from direct contact with said human skin.

6. The material of claim 1, wherein said at least one resin layer further comprises at least one structural ply layer with fiber fillers.

7. The material of claim 6, wherein said fiber fillers comprise any of carbon fibers, glass fibers, graphite fibers, polymeric fibers, aramide fibers, plant fibers, carbon nanofibers, and carbon nanotubes.

8. The material of claim 1, further obtaining a cosmetic cap layer, and further consolidating said cosmetic cap layer on a side of said at least one resin layer opposite to said clinical cap layer.

9. The material of claim 8, wherein said cosmetic cap layer is not intended for direct contact with said human skin, and wherein said cosmetic cap layer is chosen for its cosmetic properties, UV resistance properties or ability to improve thermoplastic material resistance to sag during a melt phase of a subsequent vacuum forming process;
and wherein said cosmetic cap layer further comprises at least one of a polyimide containing film, thermoplastic poly olefin containing film, biaxially-oriented polypropylene film, or other polypropylene containing film.

10. The material of claim 8, wherein said cosmetic cap layer has a thickness between 2/1000 and 12/1000 of an inch and wherein said cosmetic cap layer is consolidated with said at least one resin layer by any of a co-extrusion, heat melt lamination, heat adhesive lamination process, or other adhesive lamination process.

11. The material of claim 1, wherein said at least one resin layer comprises a plurality of structural plies.

12. The material of claim 11, wherein at least some of said structural plies comprise either a mixture of both resin and fibers or a carbon fiber veil or carbon fiber veil reminant ply.

13. The material of claim 1, wherein said at least one resin layer comprises a material selected from the group consisting of Acrylonitrile Butadiene Styrene (ABS) resin, acrylic/PVC thermoplastic sheets (Kydex), polycarbonate resin (Lexan), co-polyester sheets (VIVAK), thermoplastic elastomer (TPE) resins, polyvinyl chloride (PVC) resins, polyolefin resins, polypropylene resins, isotatic polypropylene resins, polystyrene, homopolymer polypropylene, co-polymer polypropylene, polyethylene, and other thermoplastic materials.

14. The material of claim 1, further constructing an orthotic device or a prosthetic device for a patient using said composite material.

15. The material of claim 1, wherein said first wall thickness is between 3/64 inches and ¾inches, and said preset specification thickness is between 75% to 125% of said first wall thickness.

16. A method of constructing a thermoplastic composite material with improved compatibility with human skin, said method comprising:
obtaining a clinical cap layer with at least one skin-side cap layer surface chosen for suitability for direct contact with human skin;
obtaining at least one resin layer comprising polymeric resin, said at least one resin layer chosen for at least an ability to undergo flow and/or draw during vacuum thermoforming so that it can be drawn from a flat sheet with a first wall thickness into a three dimensional shape with a double horizon bend while maintaining a second wall thickness greater than a preset specification thickness;
consolidating said clinical cap layer with said at least one resin layer so as to provide a thermoplastic composite with at least one skin-side cap layer surface chosen for suitability for direct contact with human skin, wherein said thermoplastic composite also provides an ability to undergo flow and/or draw during vacuum thermoforming so that it can be drawn from a flat sheet with a first wall thickness into a three dimensional shape with a double horizon bend while maintaining a second wall thickness greater than a preset specification thickness.

17. The method of claim 16, wherein:
a) said clinical cap layer has a thickness between 2/1000 and 12/1000 of an inch;
b) said clinical cap layer comprises a material selected for its abilities for at least one of reducing skin irritation, low friction with skin, cushioning (durometer) value, hypoallergenic properties, conductivity, or antimicrobial activity;
c) and wherein said clinical cap layer further comprises at least one of a polyimide containing film, thermoplastic poly olefin containing film, biaxially-oriented polypropylene film, or other polypropylene containing film.

18. The method of claim 16, wherein said material further comprises a cosmetic cap layer on a side of said at least one resin layer opposite to said clinical cap layer; and wherein:
a) cosmetic cap layer is selected for its abilities for at least one of cosmetic properties, UV resistance properties or ability to improve thermoplastic material resistance to sag during a melt phase of a subsequent vacuum forming process;
b) wherein said cosmetic cap layer further comprises at least one of a polyimide containing film, thermoplastic poly olefin containing film, biaxially-oriented polypropylene film, or other polypropylene containing film; and
wherein said cosmetic cap layer has a thickness between 2/1000 and 12/1000 of an inch.

19. An orthotic device or a prosthetic device for a patient formed from a thermoplastic composite material with improved compatibility with human skin, said device comprising:
a clinical cap layer with at least one skin-side cap layer surface chosen for suitability for direct contact with human skin;
at least one resin layer comprising polymeric resin, said at least one resin layer chosen for at least some ability to undergo flow and/or draw during vacuum thermoforming so that it can be drawn from a flat sheet with a first wall thickness into a three dimensional shape with a double horizon bend while maintaining a second wall thickness greater than a preset specification thickness;
said at least one resin layer being consolidated with said clinical cap layer so as to provide a thermoplastic composite with at least one skin-side cap layer surface chosen for suitability for direct contact with human skin, while also providing an ability to undergo flow and/or draw during vacuum thermoforming so that it can be drawn from a flat sheet with a first wall thickness into a three dimensional shape with a double horizon bend while maintaining a second wall thickness greater than a preset specification thickness.

20. The device of claim 19, wherein:
a) said clinical cap layer has a thickness between 2/1000 and 12/1000 of an inch;
b) said clinical cap layer comprises a material selected for its abilities for at least one of reducing skin irritation, low friction with skin, cushioning (durometer) value, hypoallergenic properties, conductivity, or antimicrobial activity;
c) and wherein said clinical cap layer further comprises at least one of a polyimide containing film, thermoplastic poly olefin containing film, biaxially-oriented polypropylene film, or other polypropylene containing film.
